(19) United States
(12) Patent Application Publication
(10) Pub. No.: US 2024/0090747 A1
(43) Pub. Date: Mar. 21, 2024

(54) ENDOSCOPE DEVICE

(71) Applicant: MEDICAL INTUBATION TECHNOLOGY CORPORATION, TAOYUAN CITY (TW)

(72) Inventor: Yi-Ting LEE, TAOYUAN CITY (TW)

(21) Appl. No.: 18/328,139

(22) Filed: Jun. 2, 2023

(30) Foreign Application Priority Data
Sep. 21, 2022 (TW) 111135752

(51) Int. Cl. A61B 1/00; A61B 1/06; A61B 1/07

(52) U.S. Cl. CPC A61B 1/00096; A61B 1/0615; A61B 1/07

(57) ABSTRACT

An endoscope device includes a head tube having a front opening and a lateral opening, a front image capturing lens disposed in the head tube and facing the front opening, an optical fiber disposed in the head tube and located at one side of the front image capturing lens, a lateral image capturing lens disposed in the head tube and facing the lateral opening, and a lateral light source disposed in the head tube and arranged adjacent to the lateral image capturing lens. As such, the endoscope device of present invention uses the optical fiber to transmit lights to the front image capturing lens and uses the lateral light source to provide lights to the lateral image capturing lens, such that the overall volume can be effectively reduced and a high intensity lighting effect can be achieved, thereby improving image capture resolution and image recognition accuracy.

### BACKGROUND OF THE INVENTION

### 1. Field of the Invention

The present invention relates to endoscopes and more particularly, to an endoscope device that has a small size and provides high intensity illumination.

### 2. Description of the Related Art

To make up for the lack of light, a transitional endoscope is provided with a plurality of light-emitting elements around an image capturing lens. For example, both TWI764057B and US2013/0274551A1 have disclosed related characteristics. However, too many light-emitting elements will make it difficult to miniaturize the size of the endoscope. It is easy to make users feel uncomfortable during operation, and further, it cannot be used in smaller body parts. Thus, there are many restrictions on the aforesaid prior arts.

### SUMMARY OF THE INVENTION

It is a primary objective of the present invention to provide an endoscope device, which can miniaturize its size and provide a high intensity lighting effect.

To attain the above objective, the endoscope device of the present invention comprises a head tube having a front opening and a lateral opening, a front image capturing lens disposed in the head tube and facing the front opening, an optical fiber disposed in the head tube and located at one side of the front image capturing lens, a lateral image capturing lens disposed in the head tube and facing the lateral opening, and a lateral light source disposed in the head tube and arranged adjacent to the lateral image capturing lens.

It can be seen from the above that the endoscope device of present invention uses the optical fiber to transmit lights to the front image capturing lens on one hand, and on the other hand, the endoscope device of present invention uses the lateral light source to provide lights to the lateral image capturing lens. As such, the overall volume can be effectively reduced and a high intensity lighting effect can be achieved so as to improve image capture resolution and image recognition accuracy.

Preferably, the optical fiber has one end thereof connected to a light-emitting element disposed outside the head tube for transmitting the lights generated by the light-emitting element to the front image capturing lens. Since the light-emitting element is not set in the head tube, there is no need to increase the size of the head tube to accommodate the light-emitting element, such that an effect of miniaturization can be achieved.

Preferably, a lens is disposed in the head tube and arranged adjacent the front image capturing lens and one end of the optical fiber for diffusing and homogenizing the lights transmitted by the optical fiber, such that the lighting effect can be enhanced.

Preferably, the lens is engaged with one end of the optical fiber through a concavity for providing a positioning effect to the optical fiber.

Preferably, the front image capturing lens is disposed to a first flexible printed circuit board. The lateral image capturing lens is disposed to a rigid printed circuit board. The lateral light source is disposed to a second flexible printed circuit board. The first flexible printed circuit board, the rigid printed circuit board, and the second flexible printed circuit board are disposed in the head tube. In this way, a simple and stable support structure is formed by the first flexible printed circuit board, the rigid printed circuit board, and the second flexible printed circuit board to be beneficial to miniaturization of the overall volume and the reduction of wear rate of components.

Preferably, a front projection lens is disposed in the head tube and arranged in a side-by-side manner with the front image capturing lens. A pattern is projected by the front projection lens towards the front opening and captured by the front image capturing lens. A lateral projection lens is disposed in the head tube and arranged in a side-by-side manner with the lateral image capturing lens. Another pattern is projected by the lateral projection lens towards the lateral opening and captured by the lateral image capturing lens. In this way, the patterns are captured by the front image capturing lens and the lateral image capturing lens to perform arithmetic operations, thus measuring the sizes of photographed objects.

Preferably, the front image capturing lens is disposed to a first flexible printed circuit board. The lateral image capturing lens is disposed to a rigid printed circuit board. The lateral light source, the front projection lens, and the lateral projection lens are disposed to a second flexible printed circuit board. The first flexible printed circuit board, the rigid printed circuit board, and the second flexible printed circuit board are disposed in the head tube. In this way, a simple and stable support structure is formed by the first flexible printed circuit board, the rigid printed circuit board, and the second flexible printed circuit board to be beneficial to miniaturization of the overall volume and the reduction of wear rate of components.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a perspective view of an endoscope device of a first embodiment of the present invention.

FIG. 2 is an exploded view of the endoscope device of the first embodiment of the present invention.

FIG. 3 is a top view of the endoscope device of the first embodiment of the present invention.

FIG. 4 is a partially sectional view of the endoscope device of the first embodiment of the present invention.

FIG. 5 is another partially sectional view of the endoscope device of the first embodiment of the present invention.

FIG. 6 is a perspective view of the endoscope device of a second embodiment of the present invention.

FIG. 7 is an exploded view of the endoscope device of the second embodiment of the present invention.

FIG. 8 is a top view of the endoscope device of the second embodiment of the present invention, in which the lens is shown as a sectional view.

FIG. 9 is a partially sectional view of the endoscope device of the second embodiment of the present invention.

FIG. 10 is another partially sectional view of the endoscope device of the second embodiment of the present invention.

### DETAILED DESCRIPTION OF THE INVENTION

First of all, it is to be mentioned that the directions used in the following embodiments and the appendix claims are based on the directions in the appendix drawings. Further, same or similar reference numerals used in the following embodiments and the appendix drawings designate same or similar elements or the structural features thereof.

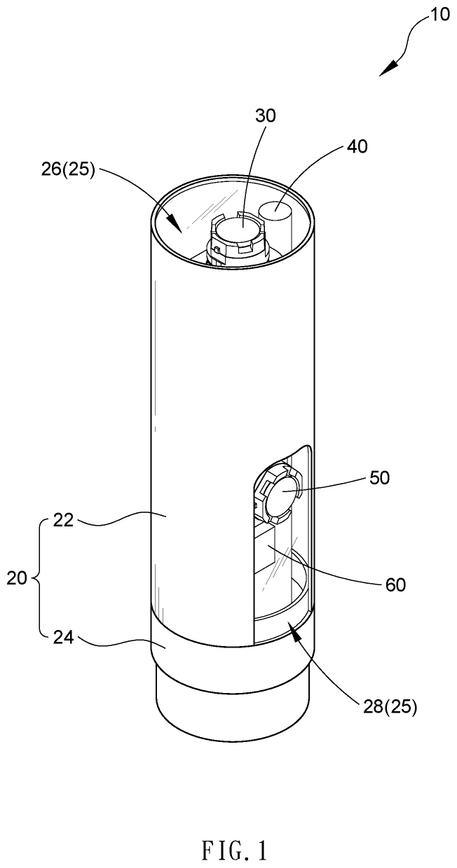

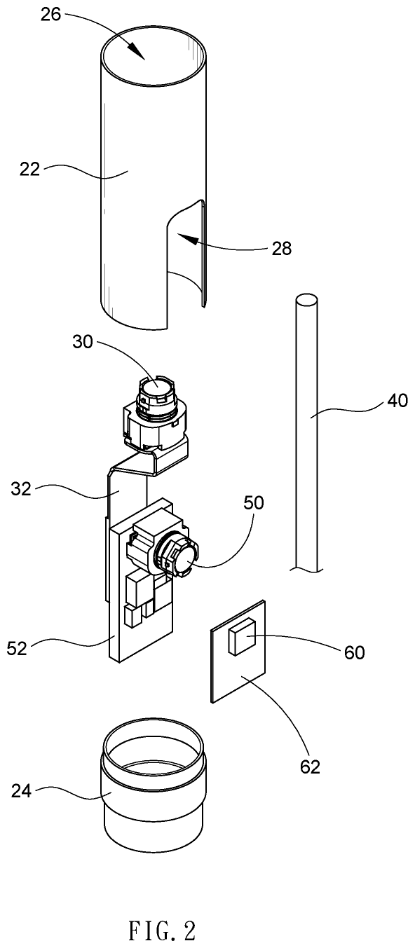

Referring to FIGS. 1 and 2, an endoscope device 10 of the first embodiment of the present invention comprises a head tube 20, a front image capturing lens 30, an optical fiber 40, a lateral image capturing lens 50, and a lateral light source 60.

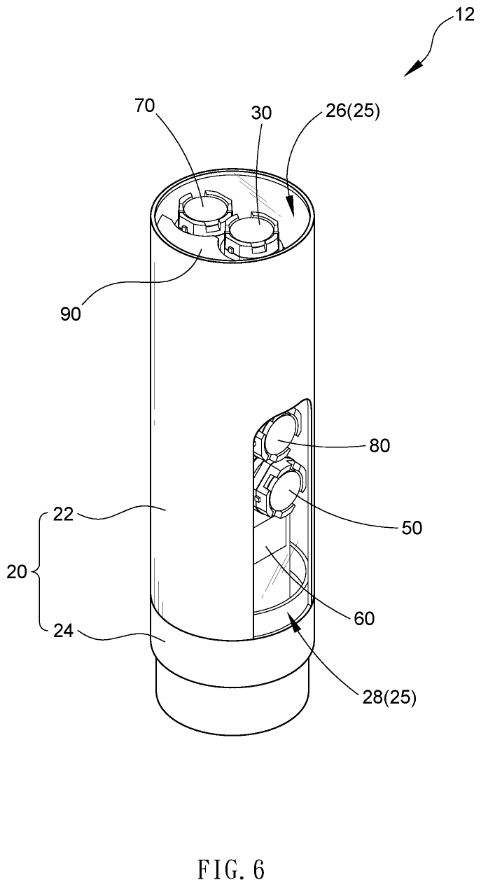

The head tube 20 is combined by an upper sleeve 22 and a lower sleeve 24. The upper sleeve 22 has a front opening 26 at a front end thereof and a lateral opening 28 at a lateral side thereof. Each of the front opening 26 and the lateral opening 28 is closed by a transparent material 25 (such as a transparent glue or transparent cover, which is not limited here). However, for convenience of illustration, the transparent material 25 is omitted and not shown in the drawings other than FIGS. 1 and 6.

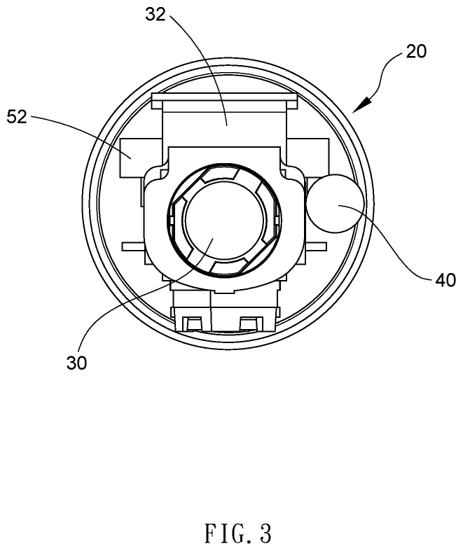

As shown in FIGS. 3 to 5, the front image capturing lens 30 is disposed in the head tube 20 and faces the front opening 26 and electrically connected to a first flexible printed circuit board 32 disposed in the head tube 20.

As shown in FIGS. 3 to 5, the optical fiber 40 is disposed in the head tube 20 and located at one side of the front image capturing lens 30 and staggered with the lateral opening 28 of the head tube 20. In addition, one end of the optical fiber 40 is adjacent to the front image capturing lens 30 and does not protrude outside the head tube 20. The other end of the optical fiber 40 extends outside the head tube 20 to be electrically connected to a light-emitting element 42 located outside the head tube 20 (here, a LED is taken as an example), so that the optical fiber 40 transmits the lights generated by the light-emitting element 42 to the front image capturing lens 30.

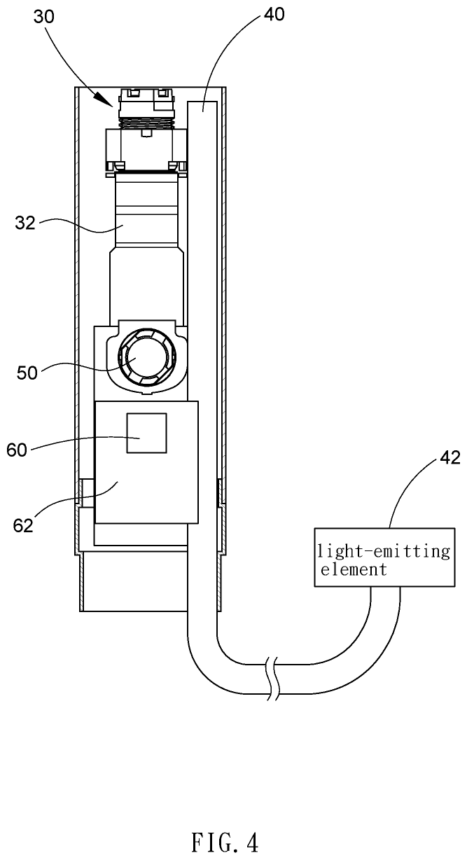

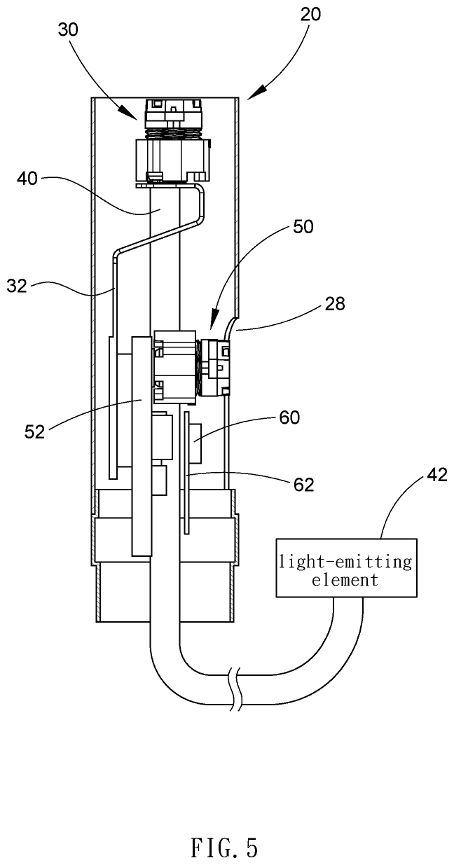

As shown in FIGS. 4 and 5, the lateral image capturing lens 50 is disposed in the head tube 20 and faces the lateral opening 28 and electrically connected to a rigid printed circuit board 52 disposed in the head tube 20.

As shown in FIGS. 4 and 5, the lateral light source 60 (here, a LED is taken as an example) is disposed in the head tube 20 and adjacent to the lateral image capturing lens 50 and electrically connected to a second flexible printed circuit board 62 disposed in the head tube 20, such that the lateral light source 60 provides lights to the lateral image capturing lens 50.

It can be seen from the above the endoscope device 10 of the first embodiment of the present invention uses the optical fiber 40 to transmit lights generated by the light-emitting element 42 to the front image capturing lens 30 on one hand, and on the other hand, the endoscope device 10 of the first embodiment of present invention uses the lateral light source 60 to provide lights to the lateral image capturing lens 50, and further, the light-emitting element 42 is located outside the head tube 20. As such, there is no need to set many light-emitting element 42 inside the head tube 20, thus achieving a purpose of miniaturization and high intensity illumination. In addition, a simple and stable support structure is formed by the first flexible printed circuit board 32, the rigid printed circuit board 52, and the second flexible printed circuit board 62 to be beneficial to miniaturization of the overall volume and the reduction of wear rate of components.

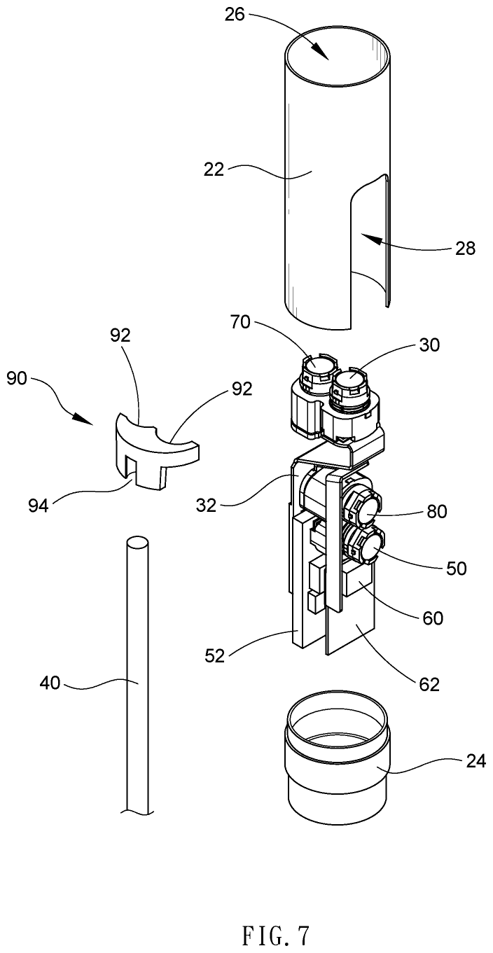

As shown in FIGS. 6 and 7, in addition to the head tube 20, the front image capturing lens 30, the optical fiber 40, the light-emitting element 42, the lateral image capturing lens 50, and the lateral light source 60, the endoscope device 12 of the second embodiment of the present invention further comprises a front projection lens 70, a lateral projection lens 80, and a lens 90.

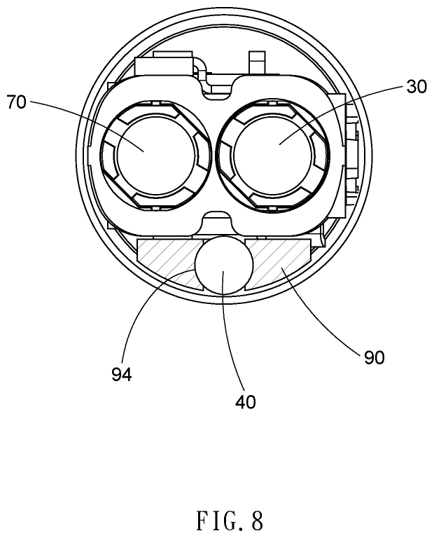

As shown in FIGS. 8 to 10, the front projection lens 70 is disposed in the head tube 20 and arranged in a side-by-side manner with the front image capturing lens 30 and electrically connected to the second flexible printed circuit board 62 together with the lateral light source 60. The front projection lens 70 uses a built-in light source (LED) to project a pattern toward the front opening 26, and then the aforesaid pattern is captured by the front image capturing lens 30 to perform arithmetic operations, thus measuring the size of a photographed object.

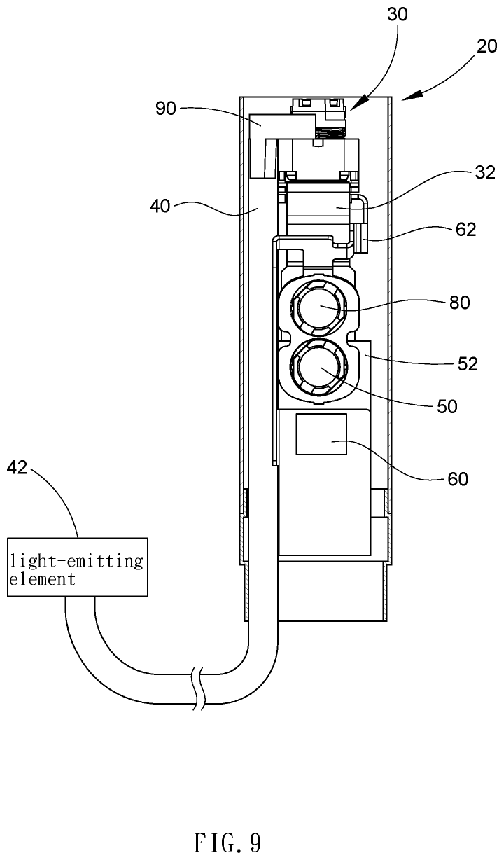

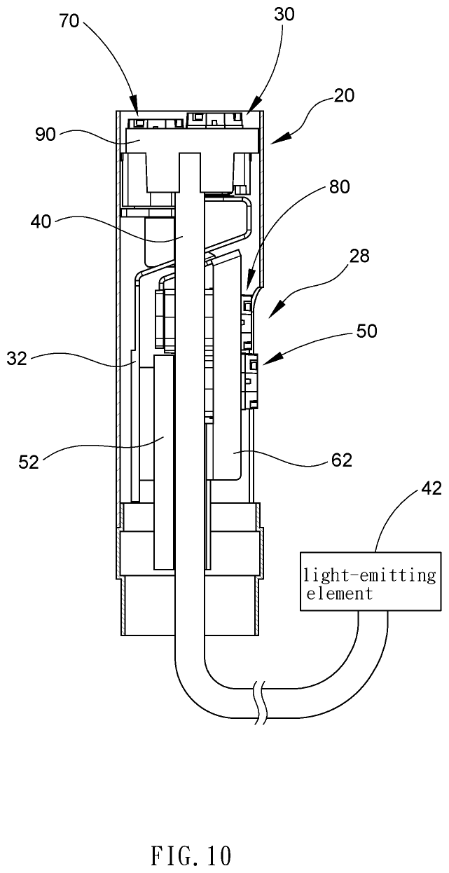

As shown in FIGS. 9 and 10, the lateral projection lens 80 is disposed in the head tube 20 and arranged in a side-by-side manner with the lateral image capturing lens 50 and electrically connected to the second flexible printed circuit board 62 together with the lateral light source 60. The lateral projection lens 80 uses a built-in light source (LED) to project a pattern toward the lateral opening 28, and then the aforesaid pattern is captured by the lateral image capturing lens 50 to perform arithmetic operations, thus measuring the size of a photographed object. What needs to be added here is that the pattern projected by the lateral projection lens 80 and the pattern projected by the front projection lens 70 are the same in this embodiment. However, in fact, the patterns projected by the front projection lens 70 and the lateral projection lens 80 are not necessarily the same.

The lens 90 is disposed in the head tube 20 and adjacent to the front image capturing lens 30 and the front projection lens 70. As shown in FIG. 7, the lens 90 has two curved grooves 92 arranged in a side-by-side manner. One of the curved grooves 92 corresponds to the profile of the front image capturing lens 30, and the other of the curved grooves 92 corresponds to the profile of the front projection lens 70. The lens 90, the front image capturing lens 30, and the front projection lens 70 are prevented from mutual interference by means of the curved grooves 92. In addition, as shown in FIGS. 7 and 8, the lens 90 further has a concavity engaged with one end of the optical fiber 40 to keep the optical fiber 40 in position. As such, when the optical fiber 40 transmits the lights generated by the light-emitting element 42 to the front image capturing lens 30, the lights are diffused and homogenized by the lens 90 to enhance a lighting effect. Moreover, since the front image capturing lens 30 uses its own light source to project the pattern, there is a need to turn off the light-emitting element 42 connected with the optical fiber 40 when the pattern is projected so as not to affect the projection effect. What needs to be added here is that the lens 90 can also be used in the first embodiment, and the configuration between the lens 90, the front image capturing lens 30, and the optical fiber 40 is the same as this embodiment, so the detail thereof is omitted herein.

As indicated above, in addition to have the same effect as the first embodiment, the endoscope device 12 of the second embodiment of the present invention uses the front image capturing lens 30 to capture the pattern projected by the front projection lens 70 and the lateral image capturing lens 50 to capture the pattern projected by the lateral projection lens 80 for performing arithmetic operations, thus measuring the sizes of the photographed objects, and further, the endoscope device 12 of the second embodiment of the present invention uses the lens 90 to diffuse and homogenize the lights transmitted by the optical fiber 40 for enhancing the lighting effect.

What is claimed is:

1. An endoscope device comprising:
a head tube having a front opening and a lateral opening;
a front image capturing lens disposed in the head tube and facing the front opening;
an optical fiber disposed in the head tube and located at one side of the front image capturing lens for providing lights to the front image capturing lens;
a lateral image capturing lens disposed in the head tube and facing the lateral opening; and
a lateral light source disposed in the head tube and arranged adjacent to the lateral image capturing lens for providing lights to the lateral image capturing lens.

2. The endoscope device as claimed in claim 1, wherein the optical fiber has one end thereof connected to a light-emitting element disposed outside the head tube for transmitting the lights generated by the light-emitting element to the front image capturing lens.

3. The endoscope device as claimed in claim 1, further comprising a front projection lens disposed in the head tube and arranged in a side-by-side manner with the front image capturing lens, and a lateral projection lens disposed in the head tube and arranged in a side-by-side manner with the lateral image capturing lens.

4. The endoscope device as claimed in claim 3, further comprising a lens disposed in the head tube and arranged adjacent the front image capturing lens and the front projection lens.

5. The endoscope device as claimed in claim 4, wherein the optical fiber has one end thereof inserted into a concavity of the lens.

6. The endoscope device as claimed in claim 3, wherein the front image capturing lens is disposed to a first flexible printed circuit board; the lateral image capturing lens is disposed to a rigid printed circuit board; the lateral light source, the front projection lens, and the lateral projection lens are disposed to a second flexible printed circuit board; the first flexible printed circuit board, the rigid printed circuit board, and the second flexible printed circuit board are disposed in the head tube.

7. The endoscope device as claimed in claim 1, further comprising a lens disposed in the head tube and arranged adjacent the front image capturing lens.

8. The endoscope device as claimed in claim 7, wherein the optical fiber has one end thereof inserted into a concavity of the lens.

9. The endoscope device as claimed in claim 1, wherein the front image capturing lens is disposed to a first flexible printed circuit board; the lateral image capturing lens is disposed to a rigid printed circuit board; the lateral light source is disposed to a second flexible printed circuit board; the first flexible printed circuit board, the rigid printed circuit board, and the second flexible printed circuit board are disposed in the head tube.
